(19) United States
(12) Patent Application Publication
(10) Pub. No.: US 2007/0093749 A1
(43) Pub. Date: Apr. 26, 2007

(54) Device for interrupting a bloodstream flowing through a cavity in an extracorporeal blood circulation circuit

(76) Inventor: Martin Spranger, Entringen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/520,443

(22) Filed: Sep. 12, 2006

(30) Foreign Application Priority Data
Sep. 13, 2005 (DE) 10 2005 045 663.4

(51) Int. Cl. A61M 29/00

(52) U.S. Cl. 604/99.01; 604/99.04

(57) ABSTRACT

The present invention relates to a device for interrupting a bloodstream flowing through a cavity, whereby the device displays a pipe-shaped cavity and a dilatable component with an aperture positioned in the cavity. Via the aperture, the at least one dilatable component is confluent with a fluid connection, whereby the at least one dilatable component is convertible from a non-dilated into a dilated state by feeding a fluid in from the fluid connection through the aperture. Moreover, mechanisms are provided in the fluid connection that control the feed-in of the fluid into the at least one dilatable component, the mechanisms being controllable via a second fluid.

### RELATED APPLICATIONS

This application claims the benefit of German application No. 10 2005 045 663.4, filed Sep. 13, 2005.

### BACKGROUND OF THE INVENTION

1. Field of the Invention

The present invention relates to a device for interrupting a bloodstream flowing through a cavity in an extracorporeal circulation circuit, with a dilatable component which can be brought from a non-dilated state into a dilated state and which interrupts the blood flow when in the dilated state.

Extracorporeal sanguiferous systems are used, for example, within the frame-work of an extracorporeal lung support in order to offer patients with a dysfunctional lung extracorporeal gas exchange.

In the case of extracorporeal circulation circuits, and especially when series-connecting appliances such as oxygenators or dialyzers, there is often a risk of gas penetrating the blood circulation system and making its way across the system of tubes into the patient's blood vessels, triggering embolisms there. In order to avoid this, fast interruption of the blood flow is necessary. To do this, the sanguiferous tubes are usually clamped as soon as it is realized that gases are being conveyed around the bloodstream in the form of bubbles.

Traditional techniques include manually or automatically actuated devices with which the blood flow through the tubes can be interrupted by exerting pressure on the external tube walls.

The primary drawback of these traditional devices—brought about by the great expenditure of force necessitated—is the delay period, which prohibits the use of such systems, given the ever shorter external retention times of the blood.

2. Related Prior Art

From European Patent EP 0 373 847 a membrane oxygenator is known, featuring a device for maintaining the pressure automatically. Among other things the device presents a valve with an internal tube, onto which pressure can be exerted from the outside. The valve further serves to stem the flow of the oxygenating gases leaving the oxygenator utilizing the pressure of the blood leaving the oxygenator. The valve disclosed here therefore serves purely to maintain a pressure with which the oxygenating gas in the oxygenator is regulated .

Thus, for instance, DE 40 12 525 discloses an actuation device for a tube system, the actuation device being able to be used optionally to block or to clear cross-sections of tubes. Also with this device, however, the tube, or alternatively the tube segments, are mechanically pressed shut, should blocking of the tube system be desired.

From EP 0 706 343 B1 a circulatory system with a blood pump and occlusion device is known, in which, via a tube, the blood is in flow connection with the blood vessel of the person to be treated. Furthermore, a sealing device is provided to regulate the flow of the blood pump. In addition, the system includes regulation mechanisms for operationalizing the obturation device in the event of a malfunction in the blood pump.

An object of the present invention is thus to provide a device with which a fluid flow, particularly in extracorporeal blood circulation circuits, can be interrupted with great speed and efficiency.

### SUMMARY OF THE INVENTION

According to the invention this object is performed by a refinement of the device mentioned by way of introduction, in which the device further displays at least one dilatable component positioned in the sanguiferous cavity, with an opening via which the at least one dilatable component is fluidly connected with a fluid connection, whereby the at least one dilatable component is convertible from a non-dilated into a dilated state by feeding a fluid in from the fluid connection through the opening, whereby moreover elements are provided in the fluid connection that control the feed-in of the fluid into the at least one dilatable component, the mechanisms being controllable via a second fluid.

Furthermore, the invention concerns a process for interrupting a bloodstream in an extracorporeal circulation circuit, whereby the device according to the invention is utilized.

Finally, the use according to the invention comprises the use of the device according to the invention for interrupting a bloodstream in an extracorporeal circulation circuit.

The object on which the invention is based is thereby performed perfectly. The device according to the invention makes it possible for the first time to effectively and very rapidly interrupt a fluid flow in a cavity system, for example the bloodstream in a system of blood tubes. System activation is effected, for instance, by means of an upstream bubble detector, which activates the device as and when required.

The invention is particularly well suited to extracorporeal blood circulation circuit tubing systems in which the bloodstream needs to be interrupted quickly in the event that gaseous or corpuscular substances are detected in the blood being returned to the human body. The device according to the invention is not confined to extracorporeal blood circulation circuits, however, but can also be utilized for any other purpose calling for the flow of a fluid to be stemmed by means of a simple and quick process.

In this case, the preferred fluid connection is a reservoir.

It is also preferable for the reservoir to be positioned outside the cavity. By having the reservoir positioned outside the cavity, the fluid can be piped out of the reservoir via an opening in the dilatable component, thus bringing the dilatable component into an expanded state and thereby blocking the flow path. The fluid with which the dilatable component is brought into the dilated state can be either a liquid or a gas of suitable viscosity.

Using the elements provided in the reservoir, the feed-in of the fluid with which the dilatable component is to be brought into a dilated state can be controlled. By controlling the fluid, it travels out of the reservoir and across the opening into the dilatable component, which widens in the process and seals the cavity.

It is particularly preferable in this respect if the volumetric quantity of fluid contained in the reservoir is at least equal to the volumetric quantity of the dilatable component in the expanded state.

This guarantees that the dilatable component is sufficiently expanded to seal off the cavity completely. To this end, the pipe-shaped cavity can be either a tube, a connector or any other tube-shaped system.

In one embodiment it is preferable if the at least one dilatable component is a balloon. In the non-expanded state the balloon will eventually be located in the flux of the fluid flowing through the cavity system and will seal off this flow path as a result of its conversion into the dilated state.

To this end it is particularly preferred if, in the expanded state, the balloon forms a tightly sealed fit against an annular constriction in the flow channel. This guarantees that even when the flow pressure is high the fluid flux is interrupted effectively and completely. In the process, the expanded balloon positions itself with its outer casing against the annular constriction, which molds to its external shape and design, the adjacent streaming pressure supporting the sealing function.

In another embodiment, for example, the dilatable component can be a dilatable inner tube which—in the manner of a ring—is positioned in the cross section of the cavity, running around its inner wall, and when a fluid is fed in through the opening into the inner tube is subject to such expansion that the cavity system is sealed by the fully expanded inner tube and the bloodstream flowing through the cavity system is throttled.

In the case of this embodiment, interruption to the cavity system and interruption to the bloodstream subsequently take place from the outside inwards with reference to the cross section of the cavity system. In the case of the aforementioned embodiment example of the dilatable balloon, which is positioned in the cavity, blocking of the cavity and, respectively, of the flux flowing through it, takes place from the inside outwards.

The latter embodiment also guarantees that sealing of the cavity system can be undertaken more quickly, should this be desirable for instance in the case of gases penetrating the blood.

From a technical point of view it is clear that a layout is conceivable in which such expandable components are set out in a multitude of arrangements.

In a preferred embodiment the mechanisms provided in the reservoir represent a piston movably positioned in the reservoir via a second fluid.

Hence, in the case of this embodiment—if a fluid flowing in a cavity has to be interrupted—a piston can be introduced into the reservoir via a second fluid, the fluid contained in the reservoir being forced by means of displacement from the reservoir through the opening of the dilatable component into this. This embodiment has the advantage that an exactly dosed amount of fluid can be quickly carried over into the expanding component and generates an accurately definable pressure here.

It is self-evident that the piston is accommodated in the reservoir in a sealing fashion, i.e. together with the reservoir the piston limits the space for the first fluid.

In the process, the piston motion, as described, is controlled by a second fluid, which presses the piston into the reservoir. The first fluid present in the reservoir is thereby displaced into the dilatable component.

On retraction of the piston, the fluid displaced into the dilatable component by negative pressure is returned to the reservoir, whereby the dilatable component is again converted into its non-dilatable state. This enables the blood flow to be interrupted quickly and efficiently on the one hand and reliable clearance of the cavity to be achieved on the other.

In another embodiment it is preferable if the means provided in the reservoir represent a second dilatable component, which in the reservoir is to be brought from a non-dilated state into a dilated state by feeding the second fluid into the second dilatable component.

In the case of this embodiment, therefore, the second fluid is introduced into the second dilatable component provided in the reservoir. The component is thereby dilated, its volume displacing the first fluid located in the reservoir. Having thus been displaced from the reservoir, the first fluid is forced into the first dilatable component via the opening of the first dilatable component, which is fluidly connected with the reservoir by virtue of this opening, whereby the component expands to seal off the cavity. This embodiment also has the advantage of being able to achieve speedy interruption of the bloodstream through the cavity, thereby advantageously avoiding the continued conveyance of particulate gases or solid matter in the bloodstream.

To this end, the second dilatable component provided in the reservoir is positioned on a wall of the reservoir so that its dilatable part protrudes into the reservoir, simultaneously sealing it off tightly. In addition, the second dilatable component presents an opening via which it is fluidly connected with the space outside the reservoir, more particularly an inlet for the second fluid. This embodiment, too, therefore, advantageously ensures that the first fluid located in the reservoir cannot escape via locations that are not sealed. This embodiment, too, therefore, advantageously guarantees that the first fluid located in the reservoir is reliably carried over into the first dilatable component.

In a further embodiment it is preferred if the mechanisms provided in the reservoir represent a membrane, which—by feeding in the second fluid—in the reservoir is convertible from an initial position into a second position.

In the case of this embodiment, therefore, there is a membrane located in the reservoir which can span the reservoir, e.g. along its full length, thereby dividing it into an initial space, which—directly—is fluidly connected with the dilatable component via the opening, and a second space, which is fluidly connected with the second fluid. The membrane is located in an initial position when the dilatable component is in the non-dilated state. In this respect there is, in the space of the reservoir, which—directly—is fluidly connected with the dilatable component, a specific quantity of the first fluid. Owing to the reservoir's direct confluence with the dilatable component, the first fluid may thus be partly present in the dilatable component, though without converting it into an expanded state when the membrane is located in the initial position.

If the second fluid is introduced into the space of the reservoir, which is fluidly connected with the second fluid, pressure is exerted on the membrane. In the process the membrane is brought from an initial position into a second position, whereby the membrane is shifted into the initial space of the reservoir, in which the first fluid is present. The first fluid is thus ousted from the reservoir and forced into the first dilatable component via the opening in the first dilatable component, which is fluidly connected with the reservoir via this opening, with this component expanding to seal the cavity.

This embodiment also has the advantage of being able to achieve speedy interruption of the bloodstream through the cavity, thereby advantageously avoiding the continued conveyance of particulate gases or solid matter in the bloodstream.

In the present case “membrane” means any component designed in the form of a foil, film, coating or leaf which displays on the one hand elastic properties and on the other hand sufficient resilience to counteract a specific pressure generated by a fluid or to yield to it. The membrane can thus feature a similar material or can consist of this, as with the second dilatable component in the embodiment described above. It is particularly preferred if the material concerned is proof or virtually proof against water vapor.

Overall, in the case of the device according to the invention, it is further preferred if a valve is provided with which the feed-in of the second fluid is adjustable.

It is preferred, moreover, if the first fluid provided in the reservoir is selected from a series of highly viscous liquids or gases, e.g. from the series of infusion solutions, physiological salines, blood expanders, artificial blood etc.

It is further preferred if the second fluid controlling the mechanisms provided in the reservoir is selected from the series of gases or alternatively mixtures thereof, e.g. atmospheric air.

This has the advantage that if, contrary to expectations, a leak in the first dilatable component should occur, it is guaranteed that there will be no passage of harmful or toxic substances when used in the extracorporeal blood circulation circuit.

It is further preferred if the first dilatable component displays a hemocompatible material when used in extracorporeal circulation.

Any reaction of the blood transported in the cavity as a result of contact with the material is thus avoided, which in the case of non-hemocompatible materials can lead, for example, to activation of the complement.

In the case of the device according to the invention, interruption of the fluid flow can be halted by de-aerating the second balloon or retracting the piston kept in the reservoir. This will serve to ensure that the first fluid flows back into the reservoir through the opening and out of the first component dilated by the fluid. In this way—as described above—the at least one dilatable component is converted back into a non-expandable state and the path through the cavity cleared. As a result, the device according to the invention remains open to multiple applications.

The invention further relates to a process with which the bloodstream in an extracorporeal circulation can be interrupted, for which purpose the device according to the invention is used.

The device according to the invention can further be utilized to interrupt a bloodstream in extracorporeal circulation, particularly in an extracorporeal lung support with a lung-assist device.

Additional advantages are illustrated in the description and the drawings enclosed.

### BRIEF DESCRIPTION OF THE FIGURES

Examples of embodiments of the invention are displayed in the drawing and will be elucidated further below by means of a description. In the figures:

FIG. 1a shows a longitudinal section through the device according to the invention with a dilatable balloon as the dilatable first component and an additional dilatable balloon provided in the reservoir, both balloons being in the non-dilated state;

FIG. 1b shows the device from FIG. 1a, this time with both balloons in the dilated state;

FIG. 2a shows a further embodiment of the device according to the invention in the longitudinal section, whereby at least one first dilatable component is a balloon and, furthermore, a piston is provided, positioned movably in the reservoir;

FIG. 2b show the device from FIG. 2a, with the balloon in the expanded state this time as a result of the piston having been introduced;

FIG. 3a shows a further embodiment of the device according to the invention, whereby a at least one first dilatable component is a dilatable tube guided along the cross section of the cavity, and an additional dilatable balloon is provided in the reservoir, both components being in the non-dilatable state;

FIG. 3b shows he device from FIG. 3a, with both dilatable components expanded;

FIG. 4a shows yet another embodiment of the device according to the invention, whereby the dilatable component as in FIG. 3a and 3b is a tube guided along the cross section of the cavity interior and whereby—as shown in FIG. 2a and 2b—a piston is positioned movably in the reservoir;

FIG. 4b shows the device from FIG. 4a, whereby the inner tube is in the expanded state as a result of the piston having been inserted into the reservoir and as a result of the fluid originally present in the reservoir thus flowing into the inner tube;

FIG. 5a shows a further embodiment of the device according to the invention, whereby the first dilatable component is a balloon and a movable membrane is additionally provided in the reservoir, located in first position; and

FIG. 5b shows the device from FIG. 5a, whereby the fluid present in the reservoir brings the balloon into the expanded state by bringing the membrane in the reservoir from an initial position into a second position.

### DETAILED DESCRIPTION OF PREFERRED EMBODIMENTS

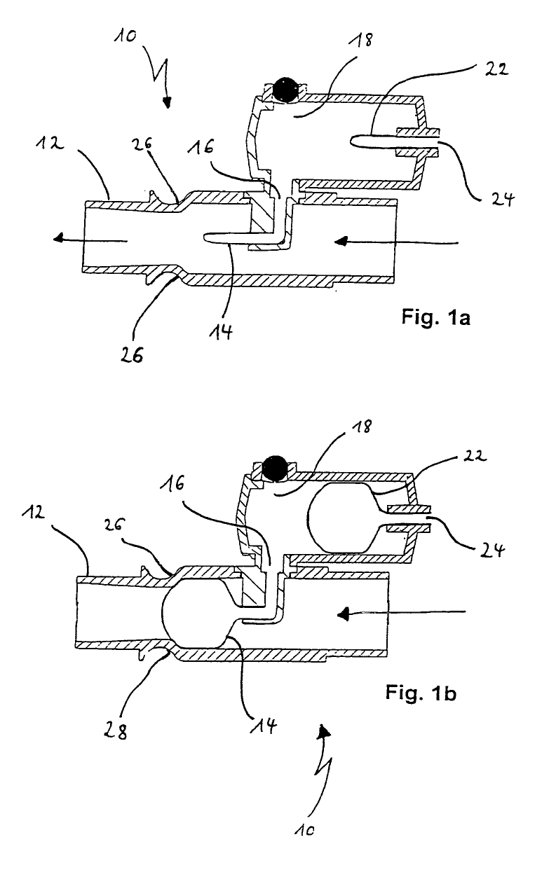

In FIG. 1a, 10 as a whole designates a device for interrupting a bloodstream flowing through a cavity 12. The device 10 displays a first dilatable element 14 in the form of a balloon, which in FIG. 1a is in the non-dilated state. The component displays an opening 16 via which it is fluidly connected with a reservoir 18. In the reservoir 18 is the first fluid, with which the dilatable element 14 can be brought into an expanded state. The device 10 further displays a second dilatable element 22, which protrudes into the reservoir 18, this element 22 being in the non-dilated state in FIG. 1a. The second dilatable element 22 displays an opening 24 via which it can be dilated with a second fluid.

In the device 10 in FIG. 1a, a ring-shaped (cross-sectional) constriction in the cavity 12 is further characterized by the reference sign 26, against which the dilated element 14 forms a tight seal.

In FIG. 1b the dilatable element 14 is in the dilated state, just like the dilatable element 22 in the reservoir 18.

Operation of the device according to the invention is such that if the blood-stream is to be interrupted—e.g. owing to the ingress of gas—the dilatable element 22 is put into a dilated state by the introduction of a second fluid through the opening 24. By increasing the volume of the dilatable element 22, the fluid present in the reservoir 18 is displaced through the opening 16 into the dilatable element 14. As a result of the flowing fluid, the dilatable element 14 is put into a dilated state in which the dilatable element 14 forms a tight seal against the annular projection 26, thereby sealing off the cavity 12. The fluid flow, or alternatively interruption of the same, in the device 10 is indicated in FIG. 1a and 1b with arrows.

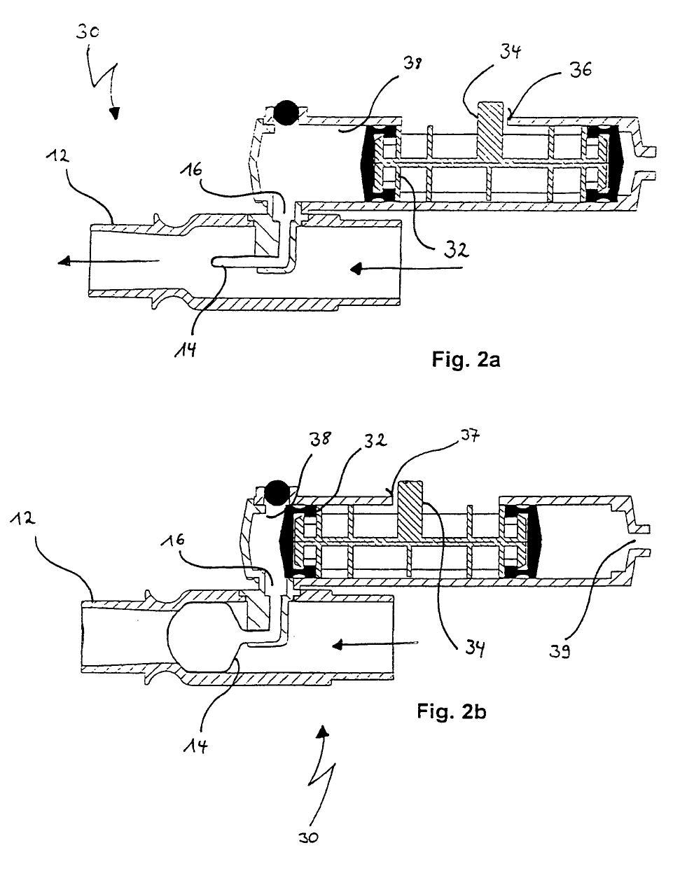

FIG. 2a shows another embodiment of the device according to the invention 30, whereby those elements that are common with elements of the embodiment from FIG. 1a and 1b are designated with the same reference signs.

This embodiment also displays a dilatable component 14 present in the cavity 12 with an opening 16 via which the dilatable component 14 is fluidly connected with a reservoir 38. The dilatable component 14 lies in the direction of flow in the cavity 12, in a non-dilated state (FIG. 2a). In the reservoir 38 a piston 32 is positioned displaceably. The piston 32 displays a pin 34 guided along a groove, via which the excursion of the piston 32 in the reservoir 38 can be limited. FIG. 2a shows an initial position of the piston 32, in which the bulk of the reservoir 38 is cleared, leaving space in the reservoir 38 for the fluid. The migration of the piston 32 out of the reservoir 38 is restricted by the pin 34, which at one point 36 of the reservoir 38 abuts against its walling and thereby stops the travel of the piston 32 out of the reservoir 38.

FIG. 2b shows the piston 32 in the reservoir 38 displaced into a second position, the fluid present in the reservoir 38 having been displaced through the opening 16 into the dilatable component 14. In the process, the dilatable component 14 is converted into an expanded state and forms a tight seal at the point 26 on the annular constriction in the cavity system 12, thereby interrupting the bloodstream in the cavity system 12. Insertion of the piston 32 into the reservoir 38 is thus limited by the pin 34 striking against a second point 37 of the reservoir 38 walling.

The piston 32, or alternatively its insertion, is thus controlled via a second fluid, which is fed in and siphoned off via the aperture 39.

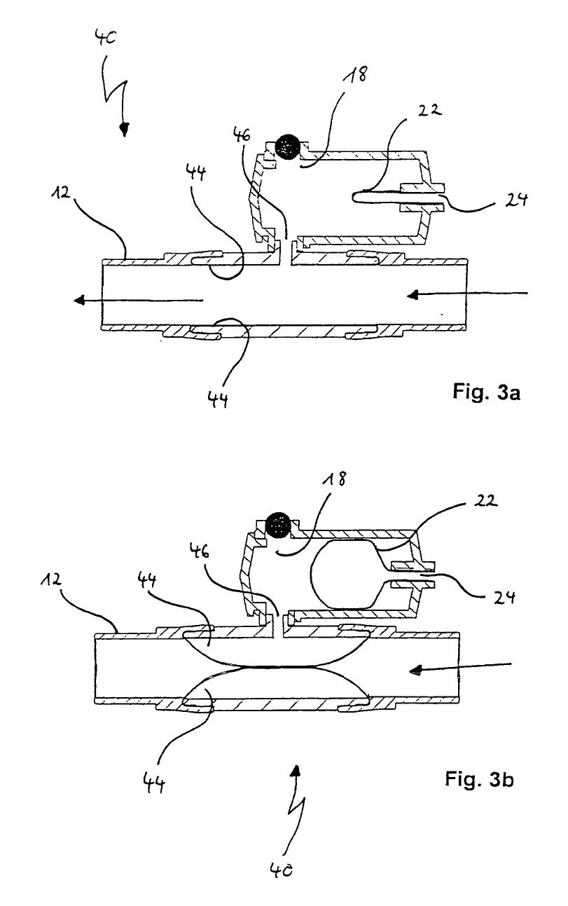

FIG. 3a shows another embodiment of the device according to the invention 40, elements similar to those in FIG. 1 and 2 again having been designated by the same reference signs. The device 40 once again displays a reservoir 18 as well as a dilatable element 22 in that reservoir 18 with an opening 24 via which the second fluid can be supplied. The device 40 further displays a dilatable component 44 in the shape of a tube guided to the inner wall of the cavity 12, which in FIG. 3a clings in the non-dilated state to the inner surface of the cavity 12 along its cross section. The dilatable element 44 is connected to the reservoir 18 via an opening 46.

If the second dilatable component 22 is now put into an expanded state by feeding in a fluid via the opening 24, the fluid present in the reservoir 18 is displaced via the opening 46 into the first dilatable element 44, whereby this is once again converted into a dilated state. The dilatable element 44, present in FIG. 3 as a tube, thus seals the cavity 12 into a dilated state. This position is shown in FIG. 3b.

By siphoning the second fluid out of the dilatable element 22 in the reservoir 18, the dilatable element 22 is converted into a non-dilatable state. Through this reduction in volume, the first fluid is returned to the reservoir 18 by negative pressure, whereby the dilatable element 44 is again converted into the non-dilated state.

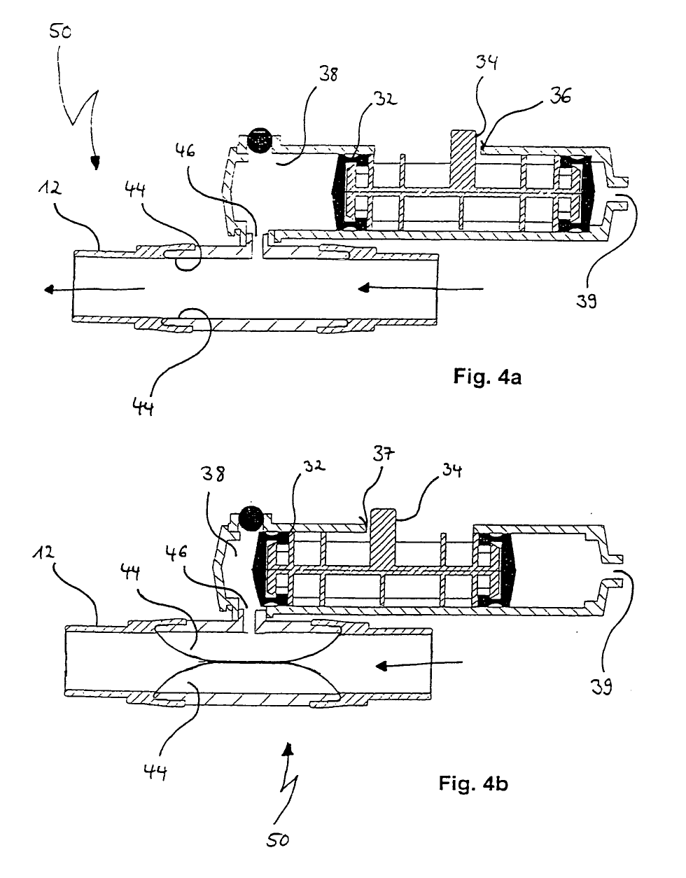

FIG. 4a shows a further embodiment of the device according to the invention 50 which, as in FIG. 3, displays a dilatable element 44 in the form of a tube guided on the inner wall of the cavity 12, shown in FIG. 4a in the non-dilated state. The dilatable element 44 in FIG. 4a clings in the non-dilated state to the inner surface of the cavity 12 along its cross section. The dilatable element 44 is fluidly connected with the reservoir 38 via the opening 46.

The reservoir 38 in FIG. 4 is fitted with a piston 32, positioned movably in the reservoir 38. As in FIG. 2 the piston 32 displays a pin 34 guided along a groove, via which the migration of the piston 32 in the reservoir 38 can be limited. FIG. 4a shows an initial position for the piston 32, in which the bulk of the reservoir 38 is cleared, thereby creating space for the fluid in the reservoir 38. The migration of the piston 32 out of the reservoir 38 is restricted by the pin 34, which at one point 36 of the reservoir 38 abuts against its walling and thereby stops the travel of the piston 32 out of the reservoir 38.

FIG. 4b shows, as in FIG. 2b, that the piston 32 in the reservoir 38 can be shifted into a second position, whereby the fluid present in the reservoir 38 is displaced into the dilatable element 44 via the opening 46, thus bringing the dilatable element 44 into an expanded state to form a tight seal at the point 26 on the annular constriction of the cavity 12, interrupting the bloodstream in the cavity 12. Insertion of the piston 32 into the reservoir 38 is thus limited by the pin 34 striking against a second point 37 of the reservoir 38 walling.

The piston 32, or insertion thereof, is thus controlled as in FIG. 2 via a second fluid, which is fed in and out via the opening 39.

By returning the piston 32 to its starting position, the first fluid is returned from the dilatable element 44 into the reservoir 38 via the opening 46 by the negative pressure arising.

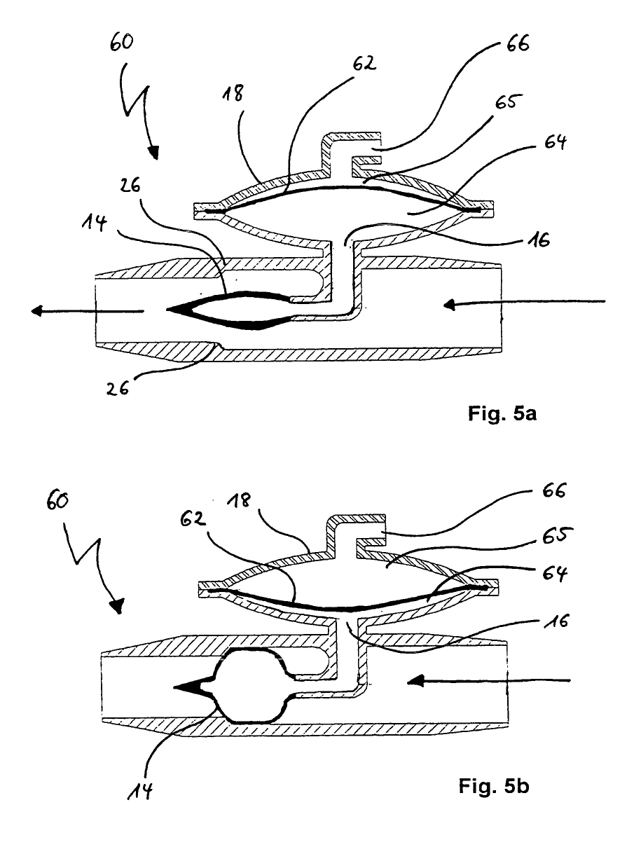

FIG. 5a shows a further embodiment of the device according to the invention 60, whereby those elements that are common with the elements of the embodiment from FIG. 1a, 1b, 2a and 2b are designated by the same reference marks.

In FIG. 5a, 60 as a whole denotes a device for interrupting a bloodstream flowing through a cavity 12. The device 60 displays an initial dilatable element 14 in the form of a balloon, which in FIG. 1a is in the non-dilated state. The element displays an opening 16 via which it is fluidly connected with a reservoir 18. Through the reservoir extends a membrane 62, spatially dividing the reservoir 18 into a first part 64 and a second part 65. The first part 64 is—directly—fluidly connected with the dilatable element 14 via the opening 16. The second part 65 is fluidly connected with the second fluid.

As in FIG. 5a the membrane 62 is in a first position whenever the dilatable element 14 is in the non-dilated state. In FIG. 5a this is shown by slight vaulting of the membrane 62 in the direction of the second part of the reservoir. The first part contains a specific amount of the first fluid. Small quantities of the first fluid may also be located in the dilatable element 14, without putting it in a dilated state, however.

In the device 60 in FIG. 5a an annular (cross-sectional) constriction in the cavity 12 is once again characterized by the reference sign 26, with which the dilated element 14 forms a tight seal.

In FIG. 5b the dilatable element 14 is in the dilated state.

Operation of the device according to the invention 60 is such that if the blood-stream is to be interrupted—e.g. on account of the ingress of gas—a second fluid is fed into the reservoir 18 via the opening 66, or alternatively into the second part 65 of the reservoir 18. By means of the second fluid, pressure is exerted on the membrane 62, which in turn transmits this pressure onto the first fluid present in the first part 64 on account of its elasticity. In the process, this in turn is forced out of the first part 64 of the reservoir 18 via the opening 16 into the dilatable element 14, causing the latter to dilate. The dilatable element 14 forms a tight seal against the circulating annular projection 26, thereby sealing off the cavity 12. The fluid flow, or interruption thereof, respectively, in the device 60 is indicated in FIG. 5a and 5b with arrows.

FIG. 5b shows the membrane arching into the first part 64 following the introduction of the second fluid into the second part 65 of the reservoir 18, whereby the volume of fluid in the first part 64 of the reservoir 18, as mentioned, is displaced into the dilatable element 14. Siphoning the second fluid out of the reservoir 18, or alternatively the second reservoir 18, “relieves” the membrane and returns it to its initial position. In the first fluid is conveyed out of the dilatable element 14 by negative pressure, the being put into a non-dilated state.

What is claimed is:

1. A device for interrupting a bloodstream flowing through a cavity in an extra-corporeal blood circulation circuit, with a pipe-shaped cavity (12) and with at least one dilatable element (14,44) positioned in the cavity (12), displaying an opening (16,46) via which at least one dilatable element (14,44) is fluidly connected with a fluid connection, whereby the at least one dilatable element (14,44) is convertible from a non-dilated into a dilated state by feeding a fluid in from the fluid connection through the opening (16,46), whereby the at least one dilatable element (14,44) interrupts the bloodstream in the dilated state, wherein moreover elements (22,32,62) are provided in the fluid connection that control the feed-in of the fluid into the at least one dilatable element (14,44), the elements (22,32,62) being controllable via a second fluid.

2. The device as claimed in claim 1, wherein the fluid connection is a reservoir (18,38).

3. The device as claimed in claim 2, wherein the volumetric quantity of fluid contained in the reservoir (18,38) is at least equal to the volumetric quantity of the at least one dilatable element (14,44) in the expanded state.

4. The device as claimed in claim 1, wherein the at least one dilatable component (14) is a balloon.

5. The device as claimed in claim 1, wherein the element is a piston (32) movably positioned in the reservoir (38) via the second fluid.

6. The device as claimed in claim 1, wherein the element is a second dilatable element (22), which in the reservoir (18) is convertible from a non-dilated state into a dilated state by feeding the second fluid into the second dilatable component (22).

7. The device as claimed in claim 1, wherein the element is a membrane (62), which in the reservoir is convertible by feeding in the second fluid from an initial position into a second position.

8. The device as claimed in claim 1, wherein a valve is further provided with which the feed-in of the second fluid is adjustable.

9. The device as claimed in claim 1, wherein the fluid contained in the reservoir is selected from the group of high-viscosity physiological liquids or gases, such as blood expanders or artificial blood.

10. The device as claimed in claim 1, wherein one dilatable element (14) displays a hemocompatible material when used in extracorporeal circulation.

11. A process for interrupting a bloodstream, wherein a device as claimed in claim 1 is utilized.

12. A process for interrupting a bloodstream in an extracorporeal blood circulation circuit to interrupt a bloodstream flowing through a cavity when gas babbles or blood clots occur, wherein a device as claimed in claim 1 is utilized.

13. A process for interrupting a bloodstream in an extracorporeal lung support with a lung-assist device, wherein a device as in claim 1 is utilized.
